(19) United States
(12) Patent Application Publication
(10) Pub. No.: US 2022/0005594 A1
(43) Pub. Date: Jan. 6, 2022

(54) Computation Model of Learning Networks

(71) Applicant: Children's Hospital Medical Center, Cincinnati, OH (US)

(72) Inventors: Michael SEID, Mason, OH (US); David HARTLEY, Rockville, MD (US)

(73) Assignee: Children's Hospital Medical Center, Cincinnati, OH (US)

(21) Appl. No.: 17/291,401

(22) PCT Filed: Nov. 5, 2019
(86) PCT No.: PCT/US19/59928

§ 371 Date: May 5, 2021

Related U.S. Application Data
(60) Provisional application No. 62/755,832, filed on Nov. 5, 2018.

(51) Int. Cl. G16H 40/20; G16H 80/00; G16H 10/60; G16H 10/20

(52) U.S. Cl. CPC G16H 40/20; G16H 10/20; G16H 10/60; G16H 80/00

(57) ABSTRACT

The present disclosure describes a computational model of a learning network. The computational model may be implemented as instructions stored on a non-transitory memory that may be executed by a processor on a local machine or as part of a cloud-based architecture employing one or more multi-thread processors enabling different users to utilize the tool to enter data and visualize results simultaneously from different locations. The computational model may accept inputs corresponding to characteristics of a patient agent and characteristics of a clinician agent. The computational model may simulate how the patient agent and the clinician agent interact with respect to a treatment selection and efficacy and may additionally and iteratively simulate further interactions between the patient agent and the clinician agent. The computational model may record how the interactions between the patient agent and the clinician agent change patient agent outcomes over time.

### BACKGROUND

How should healthcare institutions and systems optimally devote limited resources such as time, effort, and money to best improve healthcare outcomes? Until now, this question has been conceptually based on qualitative theory, subject matter expertise, and iterative, experiential learning. Predicated on the assertion that outcomes for a given population are maximized by matching each individual patient to a best treatment(s), Learning Networks (LNs) have recently been shown to improve population outcomes.

Learning Networks (LNs) typically show their theory of change in a Key Driver Diagram (as shown in FIG. 1) and devote resources to all key drivers at once. This is inefficient because it does not allow addressing pragmatic questions such as: What if we focused on one or two key drivers first? What if we only devoted half the resources to a subset of the drivers? What if one of the key drivers had to be completely finished before others would have an effect?

### SUMMARY

The current disclosure provides a computational model of a generalized LN to help answer these and related questions thus guiding strategic planning and increasing the rate of learning. Agent-based models (ABMs; sometimes known as individual-based models, IBMs) are computer programs in which artificial agents interact based on a set of rules within an environment specified by the researcher. These computational models are useful for helping decision makers think carefully about their system to explore the relations between LN actions, policies, and structure. The LN ABM disclosed herein simulates how patients and healthcare providers (e.g., doctors) interact to create and share information about what treatments are likely to work best and how these interactions change outcomes over time. By changing different factors in the model, healthcare institutions can explore what happens at different levels of Key Drivers such as access and communication; proactive, timely, and reliable care; diagnostic accuracy; appropriateness of treatment selection; or any and all combinations of these and other parameters.

The current disclosure provides an iterative modeling process in which an expert panel of patients, clinicians, and researchers is convened to refine the preliminary LN model and to explore various scenarios to provide potential answers to questions of investment currently being asked. Using the models including those disclosed herein, healthcare institutions can determine which assumptions and parameters are associated with greatest change in outcomes and how big an effect size would be necessary for a given intervention to make a difference (“sensitivity analysis”). For example, an institution might find that a campaign organizing interventions to increase patient engagement in a LN only needs 5-10,000 people aware (instead of an a priori, arbitrary goal of 25,000) in order to maximize overall participation. An institution might find that a modest, probably attainable 10% increase in the amount of sharing on a knowledge sharing platform could catalyze huge gains in knowledge. Or the institution might find that such a 10% increase only has an effect in the presence of timely, reliable care. The insights derived through a complete and systematic analysis of the model are likely to further refine strategic and operational decisions.

In an embodiment, the model has two modules. A generic core module represents the factors that determine patient-treatment matching (both the initial match and the iterative improvement of that match, i.e., ensuring that patients presenting with different conditions are administered appropriate therapy). Condition-specific modules represent the impact of patient-treatment matching on patient-level outcomes. Output from the core module is represented as ‘knowledge’ for matching patients to treatments, which serves as an input into the condition-specific module. Using this modular approach, general lessons about the functioning of LNs can be translated into condition-specific outcome curves over time.

Wagner's Chronic Care Model suggests that best outcomes arise from shared decision making within productive interactions between prepared, proactive clinical teams and informed, activated patients. Accordingly, an exemplary model according to the current disclosure is built up from iterative interactions between patient and clinician agents. In the model, patient and clinician agents meet and, based on available data (patient, clinician, and treatment attributes), determine an initial patient-treatment match. The goodness of this match is not, a priori, known. The agents have to interact again and evaluate treatment impact. Information is defined as observation of the degree to which a given treatment(s) improves outcomes for a given patient (e.g., phenotype X combined with treatment Y yields outcome Z). Based on this information, they can decide whether to continue with the current treatment or change to another. Information (about what works, for whom) can continue to reside only with the patient-clinician dyad, or it could spread. The level of knowledge is defined as the prevalence of information in a population (e.g., patients, clinicians, patient/clinician dyads). In the exemplary model, the degree to which information becomes knowledge depends on the functioning of the network. Per the Actor-Oriented Architecture, network functioning depends on the presence of sufficient actors with the will and capability to self-organize, a “commons” where actors can create and share resources, and ways to facilitate multi-actor collaboration.

In the exemplary model, parameters for actors include the number of each type of actor, initial characteristics (e.g., patient phenotype, degree to which patients are informed and activated and clinicians are prepared and proactive), the rules under which these characteristics change (e.g., patients become more active when exposed to a peer network or when interacting with a prepared, proactive clinician), and the initial network structure among and between clinicians and patients (e.g., patients are linked many-to-one to clinicians to simulate a patient panel). Parameters for the commons include how much information is available, the rate at which information generated at the point of care is captured, and the rate at which captured information is sharable. Parameters for facilitating collaboration include those governing how often patients and clinicians interact, the rules for determining how and how much information is produced at each clinical interaction (for example, an active patient and encouraging clinician create more information, while an active patient and reluctant clinician may not), the rate at which information is spread across patient-patient and clinician-clinician networks, and the rate at which information is reliably implemented into the chosen patient-treatment match.

Translation of knowledge into outcomes is tailored to specific conditions and populations, based on published evidence of treatment effects, as well as heterogeneity of the effects, and on consultation with clinical and patient subject-matter experts. The stochastic model, for each combination of the generic core parameters, is run multiple times to generate an ‘outcomes curve’ with associated confidence interval.

Exemplary embodiments can simulate the functioning of a theoretical LN under different parameter combinations. The technology can support the pragmatic design and evaluation of future LNs as well as suggest and evaluate ways to optimize existing LNs. These actions have, until now, been largely experimental and speculative in nature. This technology represents a tool for making these processes systematic, objective, and quantitative.

An embodiment of the current disclosure provides a simulation model of a learning network and a user interface to manipulate that model. The embodiment models two kinds of agents patients and clinicians. The patients may vary along several dimensions: the phenotype of their condition; the severity of their condition; their engagement; their adherence to treatment; their response to treatments; their learning from other patients; and their arrival and departure from the learning network. The clinician agents also vary along several dimensions: their engagement; their ability to correctly diagnose; their learning from patients; their learning from other clinicians; and their employment turnover. Patients and clinicians interact in several events: initial diagnosis; treatment prescription; monitoring; subsequent diagnosis; and adjustment in treatment. These events are modeled over simulated time.

The current disclosure may also include embodiments that are not limited to the healthcare setting, and embodiments may be applied to non-healthcare learning communities. For example, any community that is based on knowledge exchange and participatory behavior may be modeled based upon principles provided in the current disclosure. Exemplary models disclosed herein may be used to model, measure, build and simulate conditions that drive participation, learning and outcomes/value for any learning community (healthcare or not). Providers and buyers/users of social CRMs (such as Lithium, Jive, RightNow, Salesforce and the like) and collaborative innovation Web applications (such as Spigit, Brightidea, Hype Software, and the like) are examples. Such models may also be applicable to internal Communities of Practice (CoPs) and corporate development learning networks that exist within commercial enterprise. Such embodiments may provide an Agent-Based Model (ABM) simulating how collaborators interact to create and share information about what outcomes/solutions are likely to work best and how these interactions change outcomes/solutions over time. Such embodiments may provide a user interface allowing a user to modify key drivers and to view how such modifications change outcomes/solutions over time. Such key drivers may include (1) access to information and (2) sharing information. Such embodiments may also provide the ability to set and modify parameters for facilitating collaboration, such as those governing how often collaborators interact, the rules for determining how and how much information is produced at each interaction, the rate at which information is spread across collaborator networks, and the rate at which information is reliably implemented into the outcomes/resolutions.

### BRIEF DESCRIPTION OF THE DRAWINGS

The foregoing and other features of the present disclosure will become more fully apparent from the following description, taken in conjunction with the accompanying drawings. Understanding that these drawings depict only several embodiments in accordance with the disclosure and are, therefore, not to be considered limiting of its scope. The disclosure will be described with additional specificity and detail through use of the accompanying drawings.

In the drawings:

FIG. 1 is an example key driver diagram showing a theory of change in an example learning network;

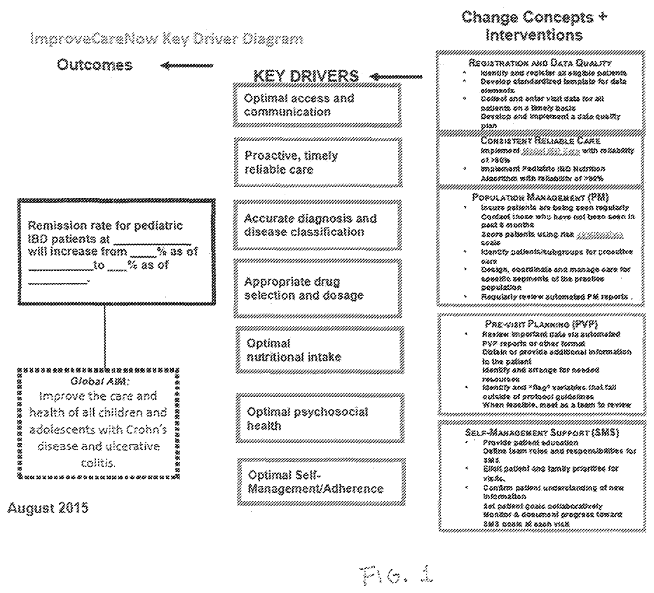

FIG. 2 is an example illustration of a computational model of a learning network according to one aspect of the present disclosure;

FIG. 3 provides an example user interface for setting up or modifying a computational model according to one aspect of the present disclosure;

FIG. 4 provides an example user interface for setting up or modifying a computational model according to another aspect of the present disclosure;

FIG. 5 provides an example user interface for setting up or modifying a computational model according to another aspect of the present disclosure;

FIG. 6 provides an example user interface for setting up or modifying a computational model according to another aspect of the present disclosure;

FIG. 7 provides an example user interface for setting up or modifying a computational model according to another aspect of the present disclosure;

FIG. 8 provides an example user interface for setting up or modifying a computational model according to another aspect of the present disclosure;

FIG. 9 provides an example user interface for setting up or modifying a computational model according to another aspect of the present disclosure;

FIG. 10 provides an example user interface for setting up or modifying a computational model according to another aspect of the present disclosure;

FIG. 11 provides an example user interface for setting up or modifying a computational model according to another aspect of the present disclosure;

FIG. 12 provides an example user interface for setting up or modifying a computational model according to another aspect of the present disclosure;

FIG. 13 provides an example user interface for setting up or modifying a computational model according to another aspect of the present disclosure;

FIG. 14 provides an example user interface for setting up or modifying a computational model according to another aspect of the present disclosure;

FIG. 15 provides an example user interface for setting up or modifying a computational model according to another aspect of the present disclosure;

FIG. 16 provides an example user interface for setting up or modifying a computational model according to another aspect of the present disclosure;

FIG. 17 provides an example user interface for setting up or modifying a computational model according to another aspect of the present disclosure;

FIG. 18 provides an example user interface for setting up or modifying a computational model according to another aspect of the present disclosure;

FIG. 19 provides an example user interface for setting up or modifying a computational model according to another aspect of the present disclosure;

FIG. 20 provides an example user interface for setting up or modifying a computational model according to another aspect of the present disclosure;

FIG. 21 provides an example user interface for setting up or modifying a computational model according to another aspect of the present disclosure;

FIG. 22 provides an example user interface for setting up or modifying a computational model according to another aspect of the present disclosure;

FIG. 23 provides an example user interface for setting up or modifying a computational model according to another aspect of the present disclosure;

FIG. 24 provides an example user interface for setting up or modifying a computational model according to another aspect of the present disclosure;

FIG. 25 provides an example user interface for setting up or modifying a computational model according to another aspect of the present disclosure;

FIG. 26 provides an example user interface for setting up or modifying a computational model according to another aspect of the present disclosure;

FIG. 27 provides an example user interface for setting up or modifying a computational model according to another aspect of the present disclosure;

FIG. 28 is an illustrating providing example graphs for visualizing selection efficiency;

FIG. 29 is an example user interface for checking whether praxis improves selection efficiency;

FIG. 30 is an example diagram plotting relationships among class entities within an example model according to the present disclosure

FIG. 31 is an example illustration of an outcome curve that may be displayed once various parameters are adjusted and run.

### DETAILED DESCRIPTION

In the following detailed description, reference is made to the accompanying drawings, which form a part hereof In the drawings, similar symbols typically identify similar components, unless context dictates otherwise. The illustrative embodiments described herein are not meant to be limiting. Other embodiments may be utilized, and other changes may be made, without departing from the spirit and scope of the subject matter presented here. It should be readily understood that the aspects of the present disclosure, as generally described herein and as illustrated in the Figures, may be arranged, substituted, combined, and designed in a wide variety of different configurations, all of which are explicitly contemplated and make part of this disclosure.

The current disclosure provides a computational model of a generalized LN to help answer these and related questions thus guiding strategic planning and increasing the rate of learning.

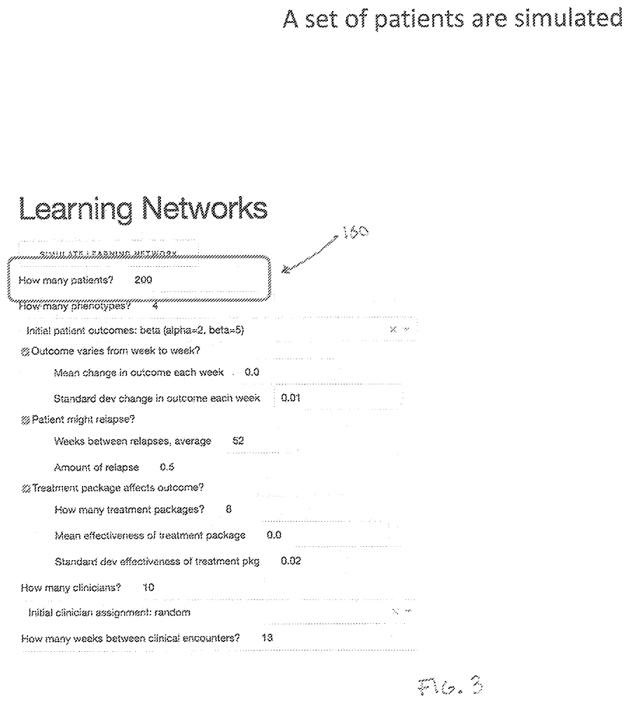

FIGS. 3-27 provide an exemplary user interface for setting up and/or modifying an exemplary computational model according to an exemplary embodiment. Generally, in FIGS. 3-29, the left-hand side of the figure will provide an image of the exemplary user interface, and the right-hand side of the figure will provide block diagram representations of the model components being set up and/or modified by the user interface or other visual guides to help explain the components being defined.

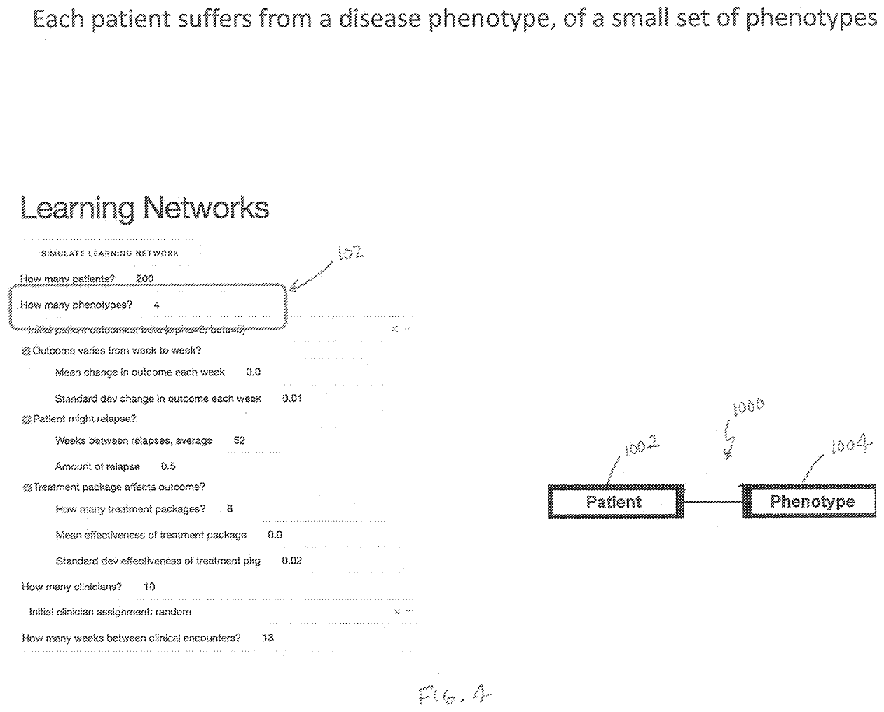

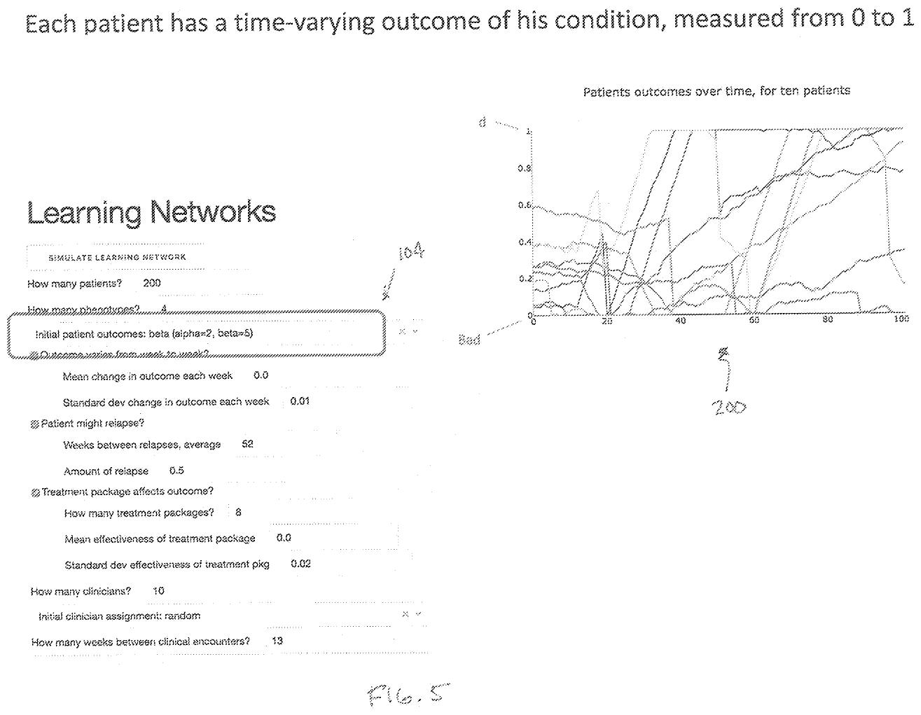

As shown in FIG. 3, a set of patients are simulated and in the user interface, and in field 100 the user specifies how many patients are simulated. Each patient suffers from a disease phenotype (i.e., type of disease distinct from other forms of the condition and to which corresponds one or more discrete types of treatments), of a small set of phenotypes, and the user specifies the number of phenotypes in user interface in field 102, as shown in FIG. 4, and the relation 1000 is that every patient 1002 suffers from exactly one phenotype 1004 and each phenotype has, in general, many patients suffering from that phenotype. As shown in FIG. 5, each patient has a time variant outcome of this condition measured in a zero to 1 scale as selected in field 104. Graph 200 in FIG. 5, illustrates an example distribution of patient outcomes over time, for ten patients.

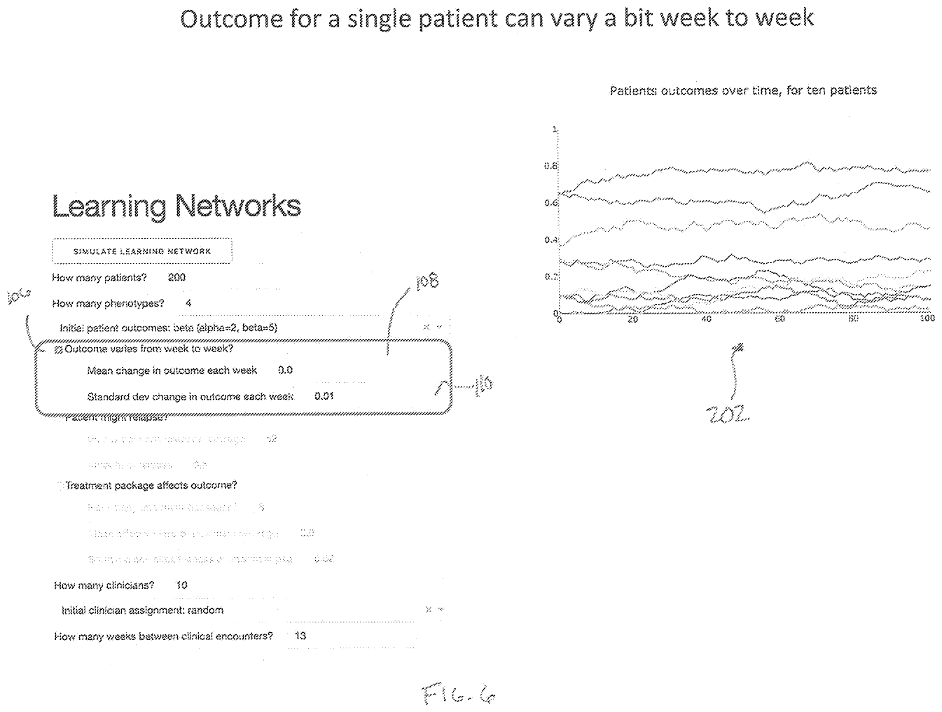

As shown in FIG. 6, there is a check-box 106 which indicates whether the patient outcomes may vary from week to week. If the box is checked, the user can define the mean change in outcome each week in field 108 and the standard deviation change in outcome each week in field 110. Graph 202 in FIG. 6 illustrates an example random distribution of patient variance of outcomes over time for ten patients.

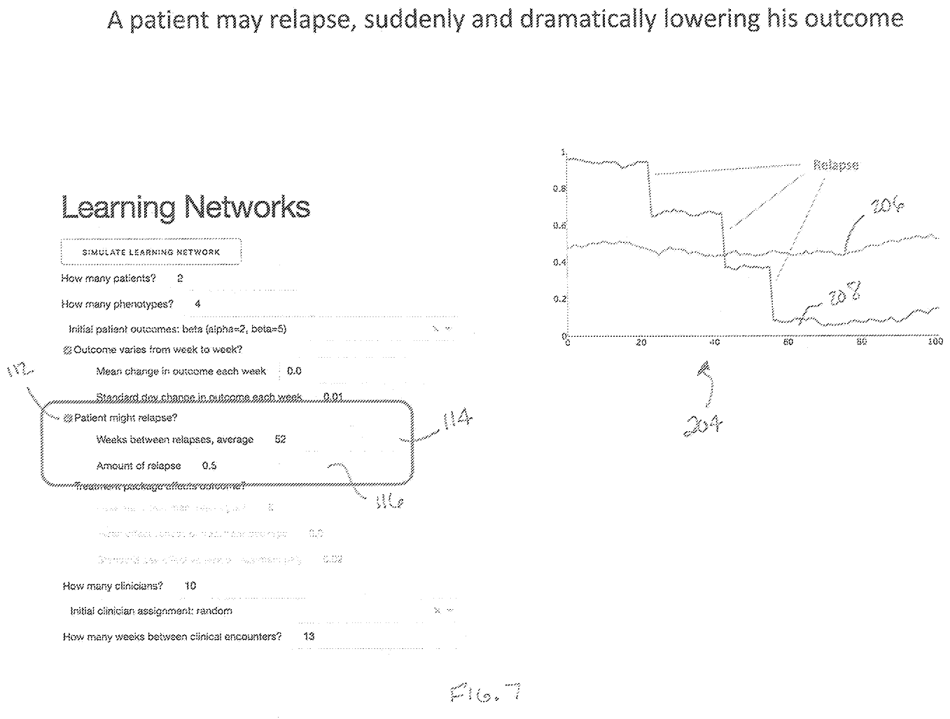

As shown in FIG. 7, a check-box 112 is provided in which a user can select whether or not the patient may relapse. If the box 112 is checked, then the user can set the average weeks between relapse in field 114 and the amount of relapse (percent effect in outcome) in field 116. A patient may relapse suddenly and dramatically, thereby lowering his/her outcome. Line 206 in graph 204 illustrates what it looks like without variation week to week and the patient doesn't experience relapse. Line 208 in graph 204 illustrates the results in outcome with a patient experiencing multiple relapses.

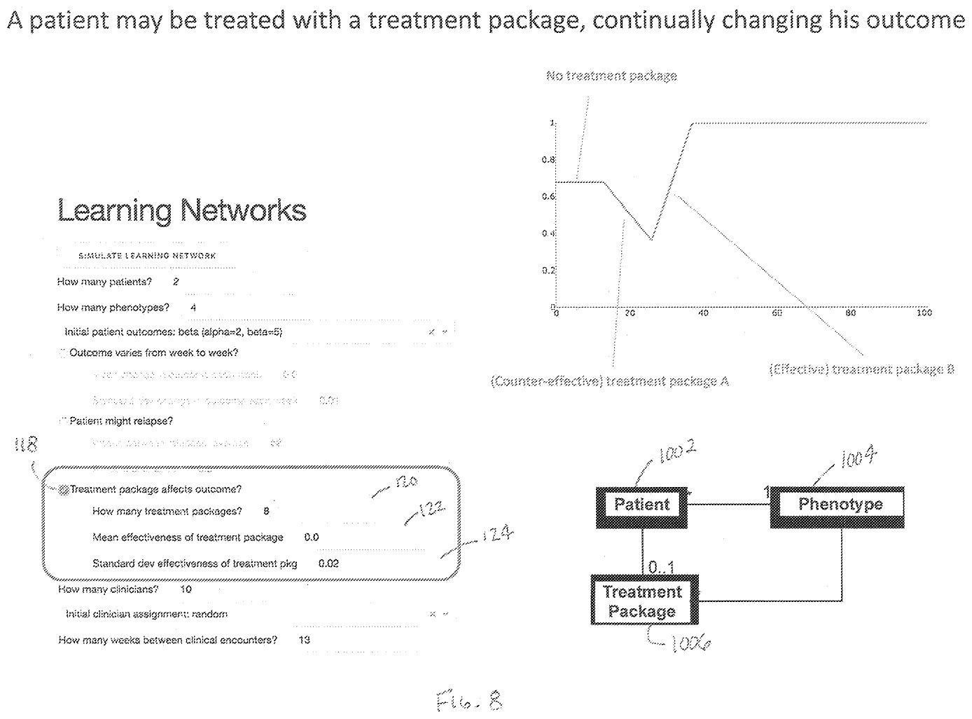

As shown in FIG. 8 a check-box 118 is provided to select whether a treatment package exists that affects outcome. A treatment package 1006 may a pharmaceutical, therapy or treatment program to treat a phenotype 1004. The treatment package may include a combination of diet, exercise, pharmaceutical and the like. As an example, a patient 1002 will have either zero or one treatment package 1006. As another example, a patient might have zero at the beginning of the simulation, and after a while, the patient will have one. Each treatment package 1006 has an effect on each phenotype 1004. There is a many-to-many relationship there between treatment package and phenotype. When box 118 is checked, the user is provided field 120 to indicate how many treatment packages are available, a field 122 to provide the mean effectiveness of the treatment package, and a field 124 to provide a standard deviation effectiveness of the treatment package. For example, if a treatment package has a 0.01 effectiveness, outcomes increase 0.01 every week for every patient that has that phenotype and is assigned that treatment package.

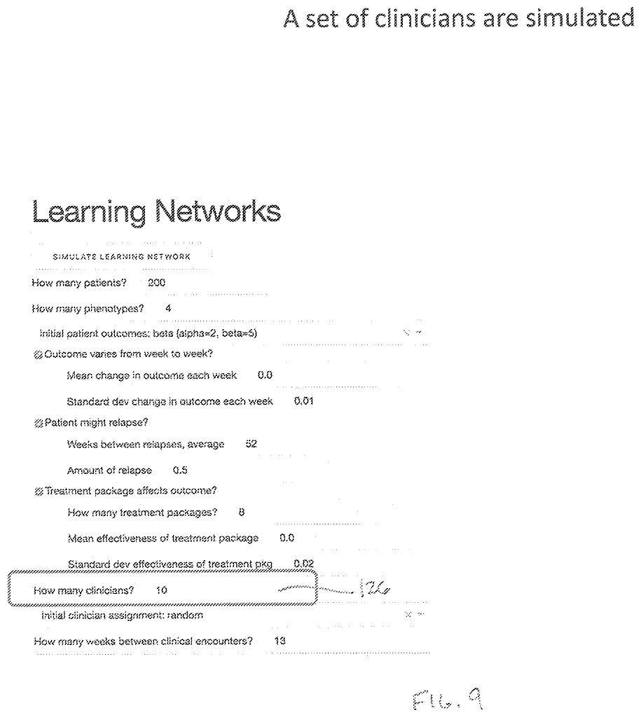

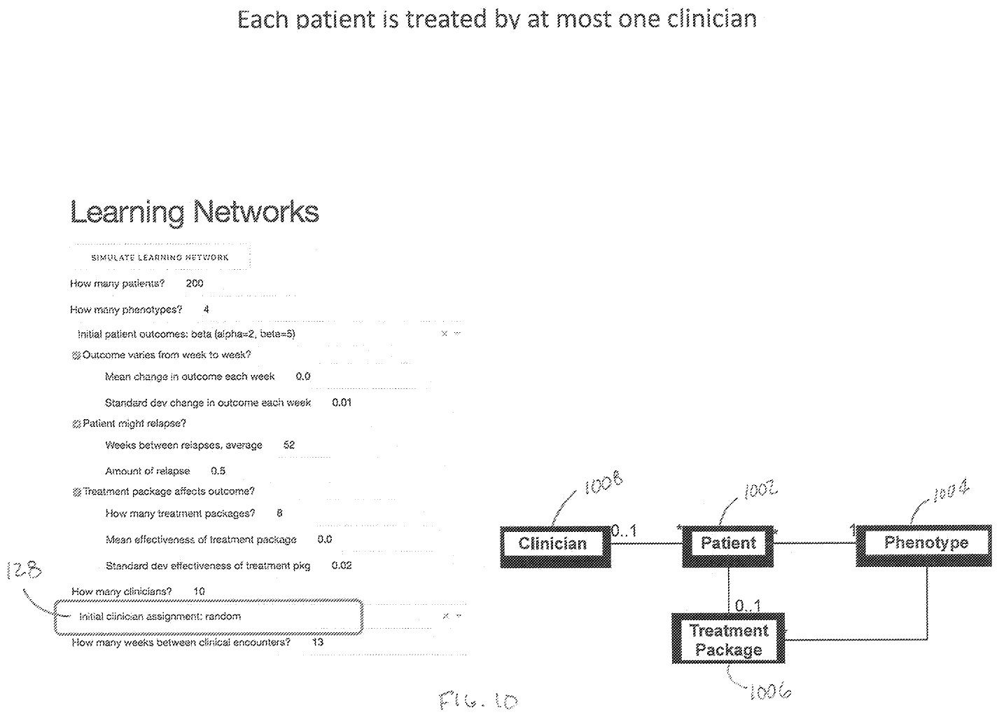

As shown in FIG. 9 field 126 allows the user to define the number of clinicians that are simulated. As shown in FIG. 10, menu 128 allows the user to select how the clinicians 1008 are assigned to patients 1002. The clinicians may be assigned randomly by default, or the clinicians may be assigned based upon a given order.

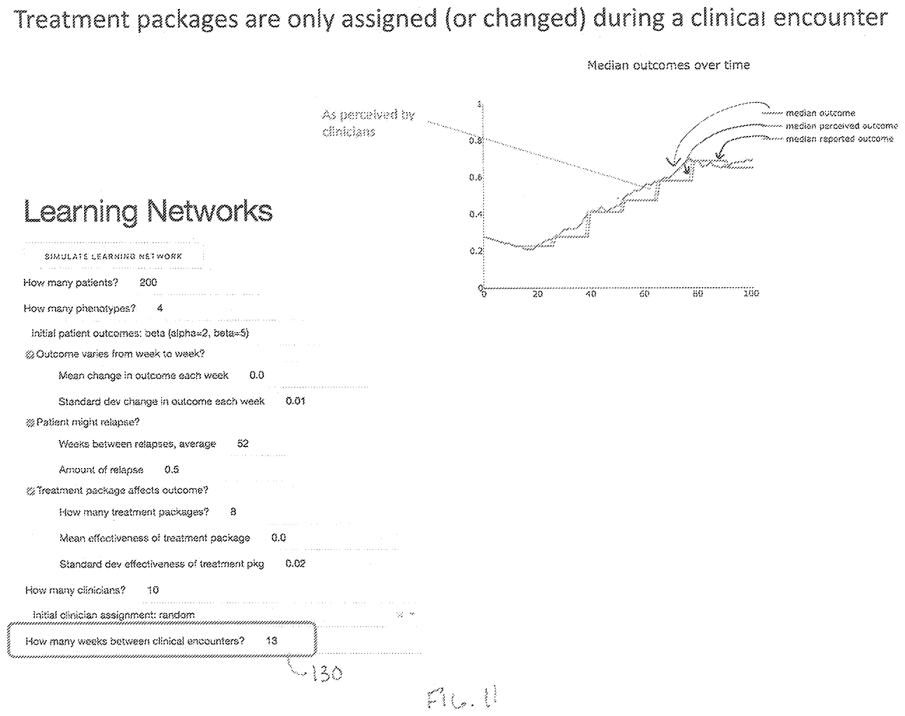

As shown in FIG. 11, a user is then provided a field 130 to set the number of weeks between the clinical encounters. For example, if the user sets 13 weeks between clinical encounters, it means for all the patients at Week 13, they see a clinician and then for all the patients at Week 26, they see a clinician. The system may also set a sort of uniform distribution between an upper bound and a lower bound. For example, one patient will be every 10 weeks, one patient will be every 16.

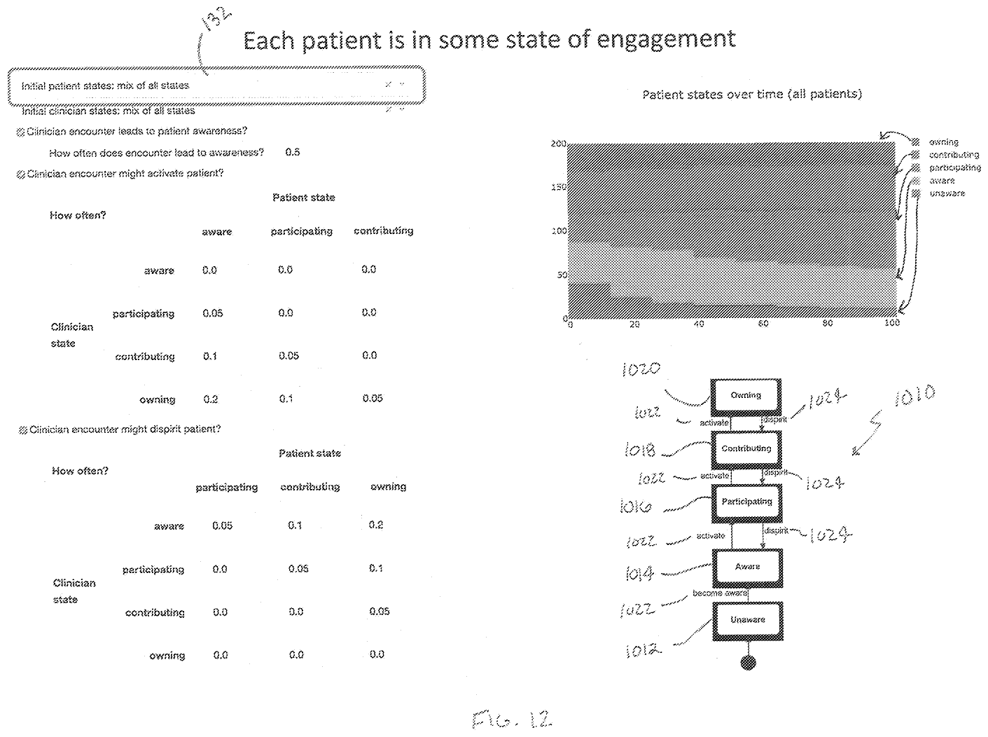

FIGS. 12-16 illustrate the user interface for setting up patient and clinician engagement/awareness with the learning network. Each patient is in some state of engagement and there are many variations for how this could be set up. As shown in FIG. 12, menu item 132 establishes the initial patient engagement state. For example, the user can select from “unaware,” “aware,” “mix of all states,” “all in succession” and “all aware in succession.”

FIG. 12 also illustrates a hierarchy of engagement 1010. “Unaware” 1012 means that the patient is unaware of the learning network; “Aware” 1014 means that the patient is aware of the learning network; “Participating” 1016 means that the patient is using the existing tools of the learning network; “Contributing” 1018 means that the patient is working to improve the tools (such as participating on chat boards); and “Owning” 1020 means that the patient is developing new solutions or inventions solving new problems according to their and the network's needs. Each of these engagement states (except for the top “Owning” state) has an ability for the patient to advance up 1022 to the next state; and each of these engagement states (except for the bottom “Unaware” and “Aware” states) has the ability for the patient to dispirit down 1024 to the previous state.

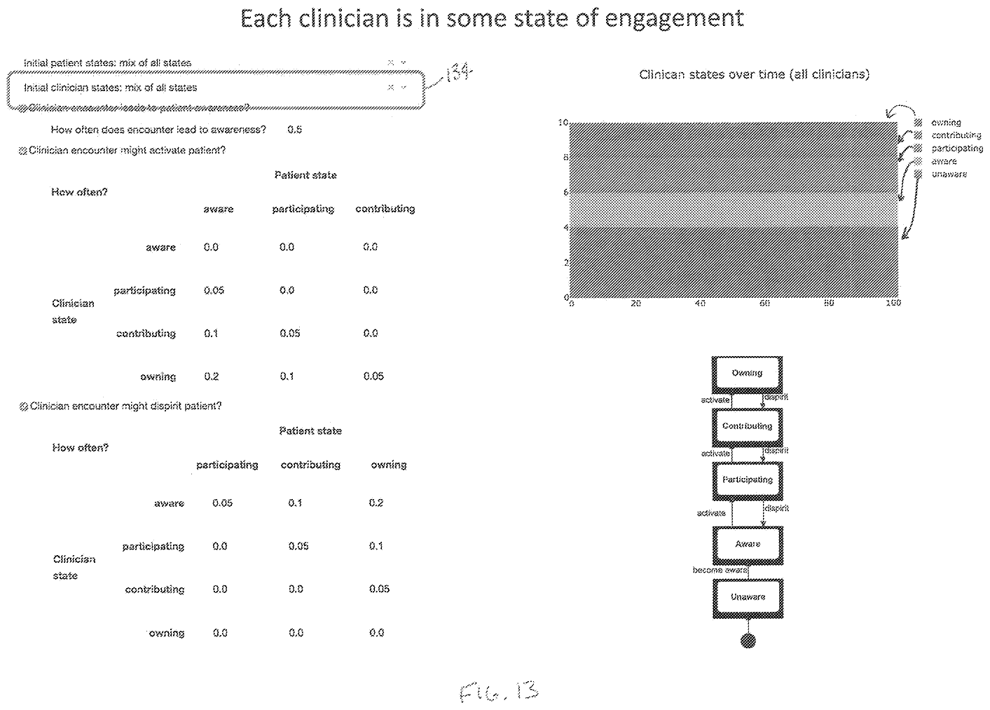

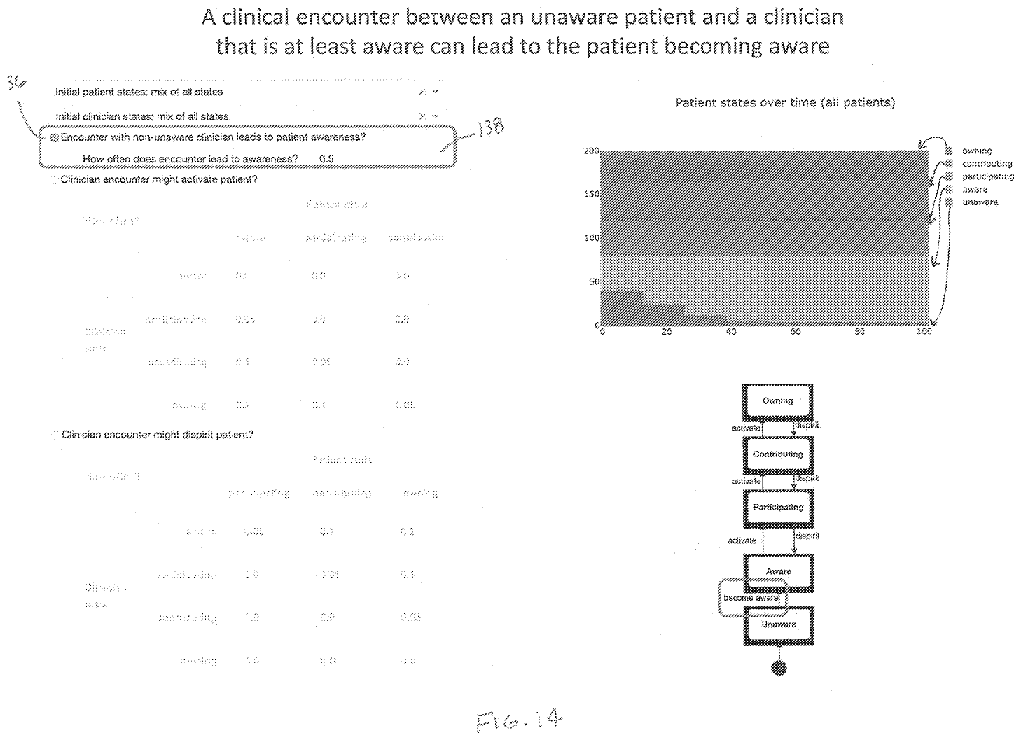

As shown in FIG. 13, menu item 134 establishes the initial clinician engagement state. As shown in FIG. 14 checkbox 136 if checked allows an un-aware patient to transition to the aware state if the patient encounters an aware clinician. Then, field 138 sets the frequency in which such an encounter leads a patient to an awareness state.

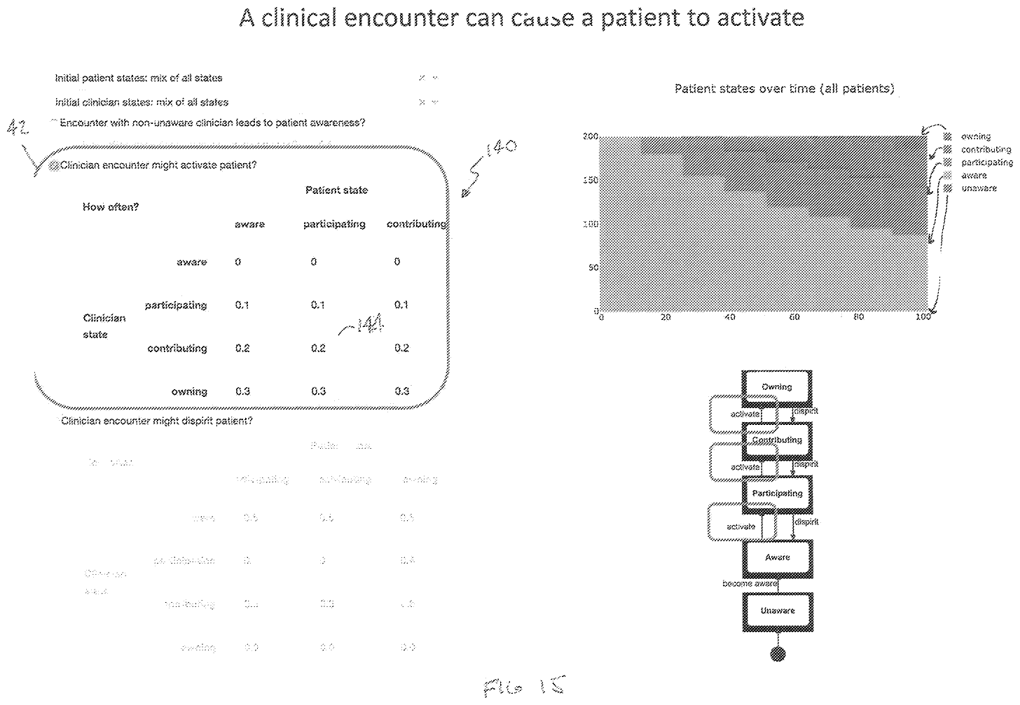

FIG. 15 shows a grid 140, activated by check-box 142, establishing how patient encounters with clinicians, depending upon their respective states of engagement, will allow the patient to activate into a higher state of engagement. The user can change what the percentages are, the probabilities are, even the clinician state and the patient state. And just for an example, as shown in box 144 in the grid 140, if a contributing clinician meets with a participating patient, there's a 0.2 (20%) chance that the patient will activate to the next level.

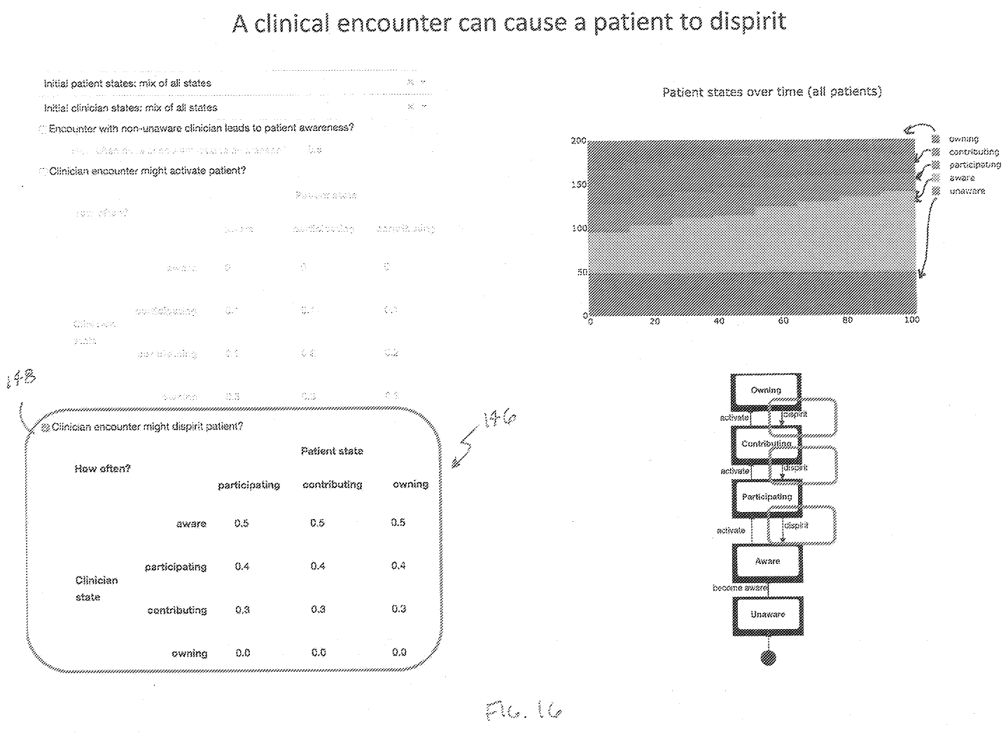

FIG. 16 shows a similar grid 146, activated by check-box 148, establishing how a patient's encounters with clinicians, depending upon their respective states of engagement, will cause the patient to dispirit to a lower engagement state.

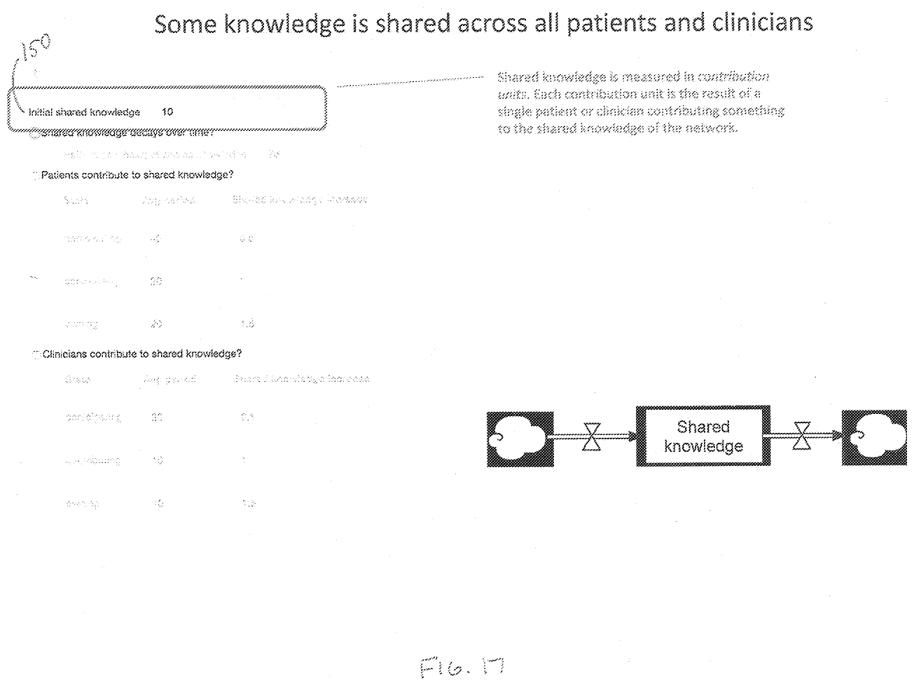

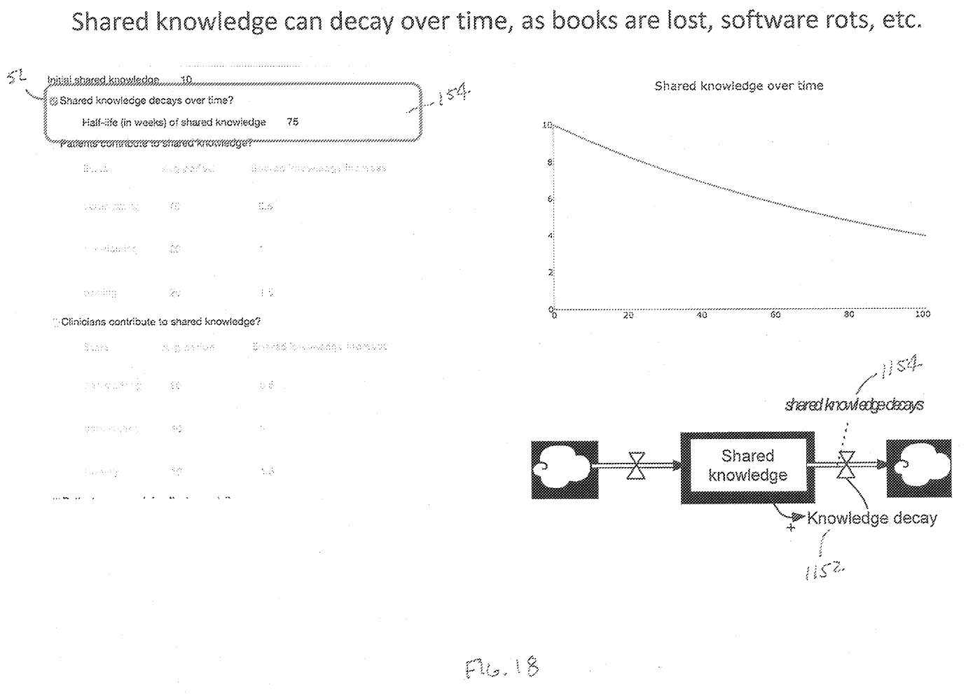

FIGS. 17-20 illustrate the user interface for setting up how knowledge is shared across all patients and clinicians. As shown in FIG. 17, a user may specify in field 150 an initial level of shared knowledge in the form of “contribution units.” Clinicians and patients can increase shared knowledge as measured in contribution units. As shown in FIG. 18, check-box 152 indicates whether the shared knowledge will decay over time. And field 154, if box 152 is checked, allows the user to provide the half-life in weeks of the shared knowledge. Examples of things or circumstances that may cause shared knowledge to decay are: people forget, software isn't maintained, practices change, and the like. In the flow diagram, activation of shared knowledge decay is illustrated by 1152 and the half-life is represented by 1154.

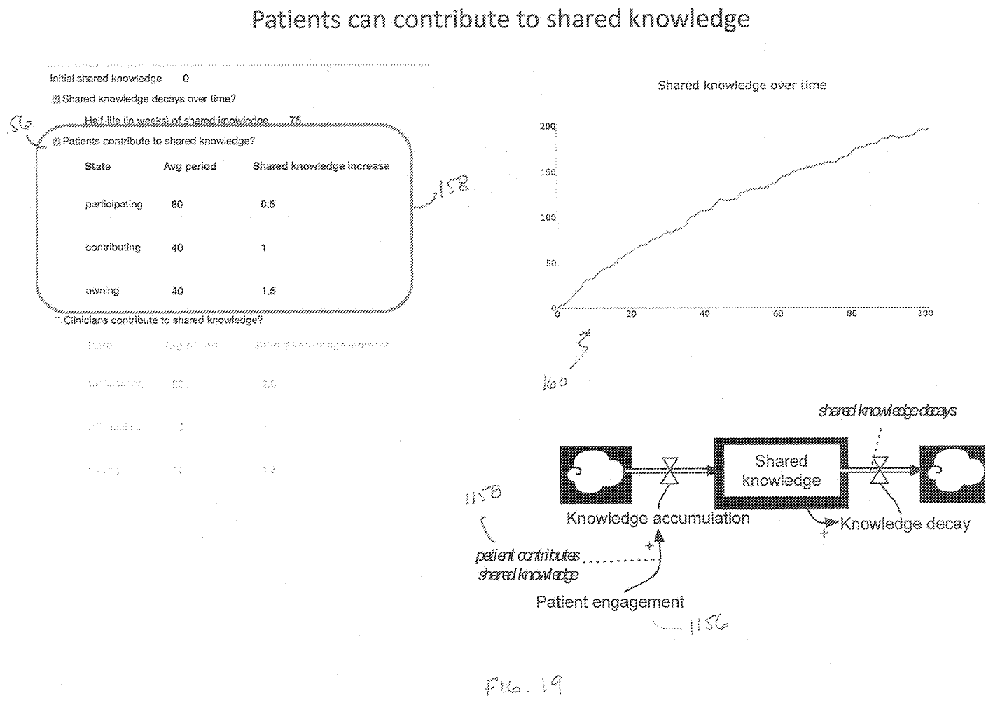

As shown by FIG. 19, check-box 156 allows control over whether patients can contribute to shared knowledge. And, if activated, grid 158 establishes, based upon the patient's engagement state, how much and how often they contribute to the shared knowledge. In the flow diagram, activation of patient shared knowledge contribution is illustrated by 1156 and the grid 158 is illustrated by 1158. Using this, the effect of patient engagement can be investigated, and illustrated by a graph 160, for example. Questions posed, for example, include ‘if there are a large number of patients and they are all adding tiny bits of knowledge, is that as good as if you have a small number of patients contributing relatively large amounts of knowledge?’ One can compare how that relates to what is being seen in the actual learning networks and then determine how to manipulate learning network from that. In the actual learning network, should it be made easier for more people to contribute smaller bits of knowledge? Or should we identify the owners and recruit them more heavily and get them to contribute more knowledge per unit? And then how does that knowledge accrue over time?

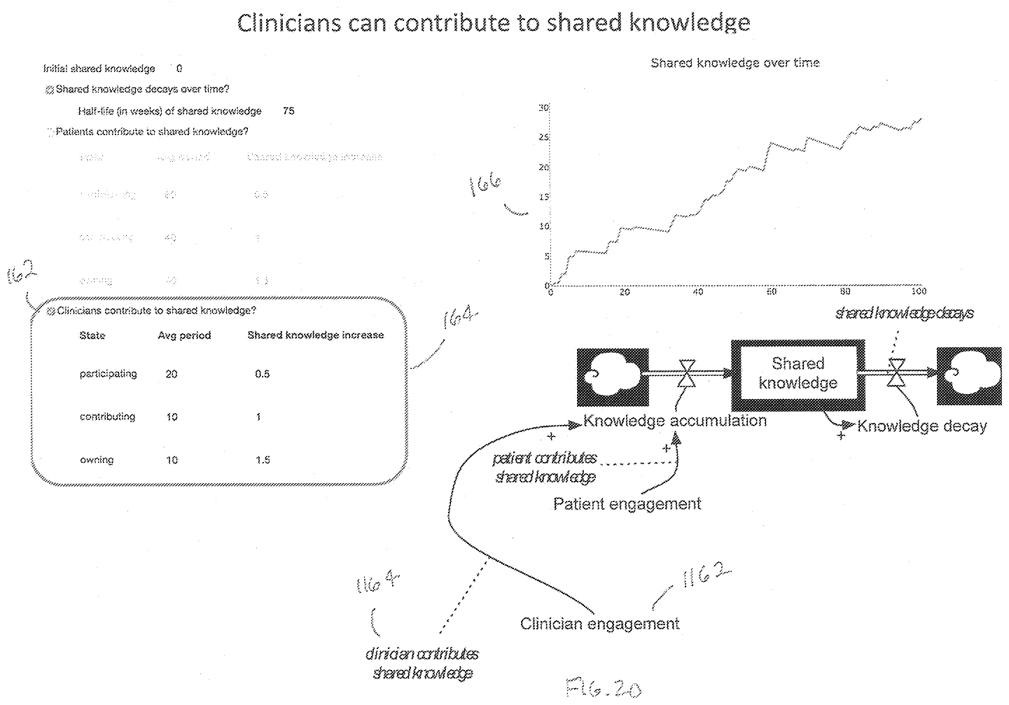

Similarly, as shown by FIG. 20, check-box 162 indicates if clinicians can contribute to shared knowledge. And, if activated, grid 164 establishes, based upon the clinician's engagement state, how they contribute to the shared knowledge. In the flow diagram, activation of clinician shared knowledge contribution is illustrated by 1162 and the grid 164 is illustrated by 1164. Using this, the effect of clinician engagement can be investigated, and illustrated by a graph 166, for example. As shown in graph 166, the clinician contributions may be larger over time than a patient contribution.

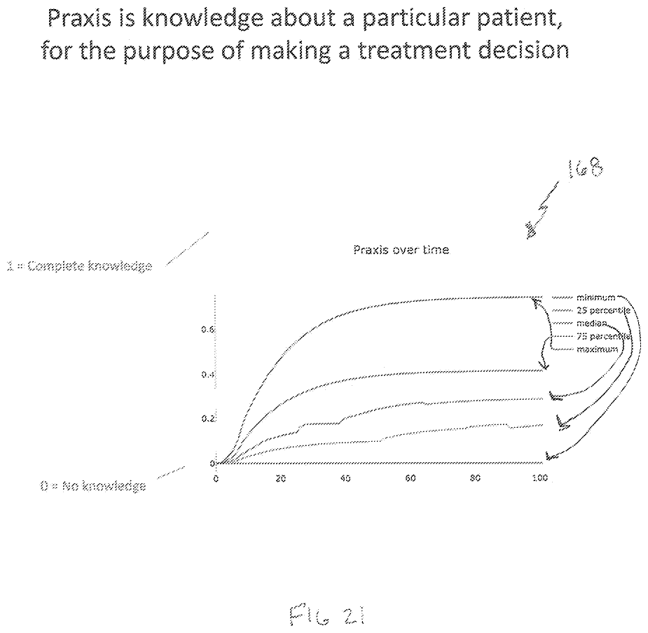

FIGS. 21-29 pertain to modeling praxis. Praxis is knowledge for a purpose, in this case, for the purpose of making a treatment decision to improve outcome for a particular patient. As shown in FIG. 21, a graph 168 showing praxis over time for a simulated network. Praxis may range from 0 (no knowledge for knowing which treatment will work best for what patient) to 1 (perfect knowledge for knowing which treatment will work best for what patient. Graph 168 shows median, minimum, maximum, 25th percentile, and 75th percentile estimates for praxis.

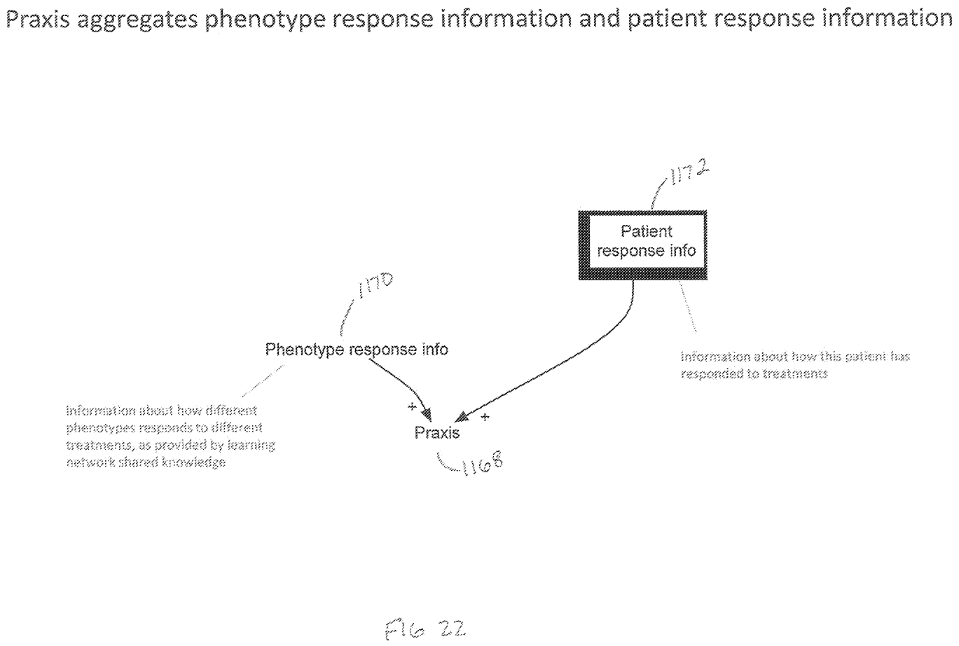

Referring to FIG. 22, praxis 1168 is a function of both “phenotype response information” and “patient response information.” Phenotype response information 1170 is the information about how different phenotypes respond to different treatments as provided by learning network shared knowledge. Patient response information 1172 is information about how this patient has responded to treatments.

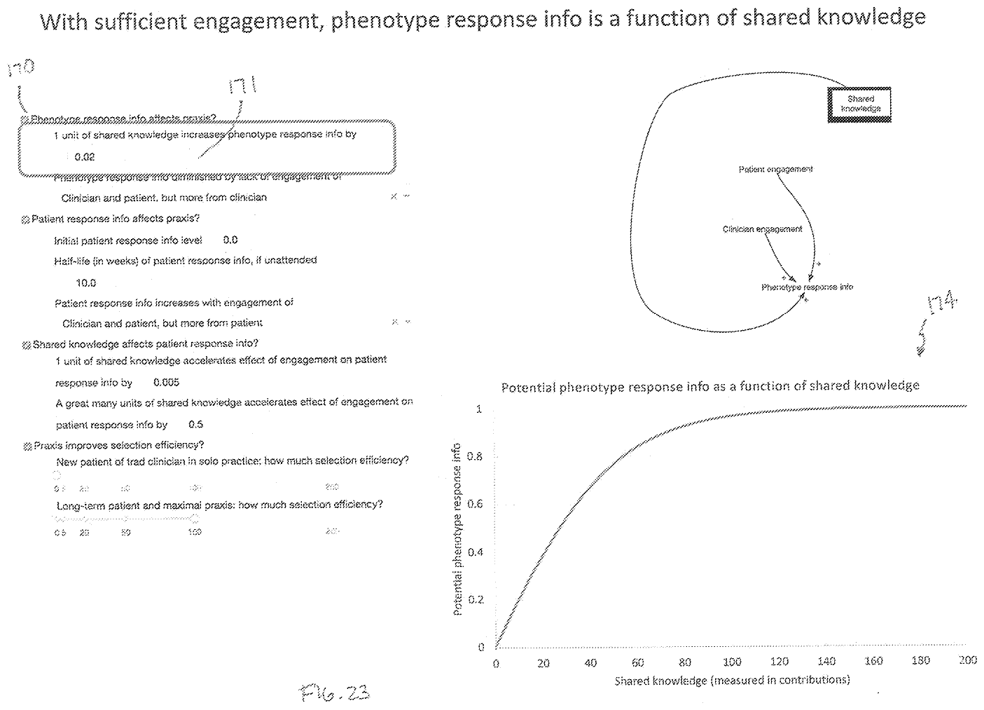

Referring to FIG. 23, a checkbox 170 in the user interface is checked if phenotype response information affects praxis. Then field 171 allows the user to provide a value for how much phenotype response information is increased by 1 unit of shared knowledge.

Phenotype response information is a function of the shared knowledge. As shown in graph 174 the more shared knowledge, the more potential phenotype response information. It's potential because it gets modulated by the patient engagement and clinician engagement. As shown in this example, the curve is controlled by the parameter in field 171 (in this case, 0.02) such that a single unit of shared knowledge will increase the potential phenotype response information by 0.02, that's the initial slope of this curve, but it tails off at one. That is one of the hypotheses that would then be tested in actual learning networks.

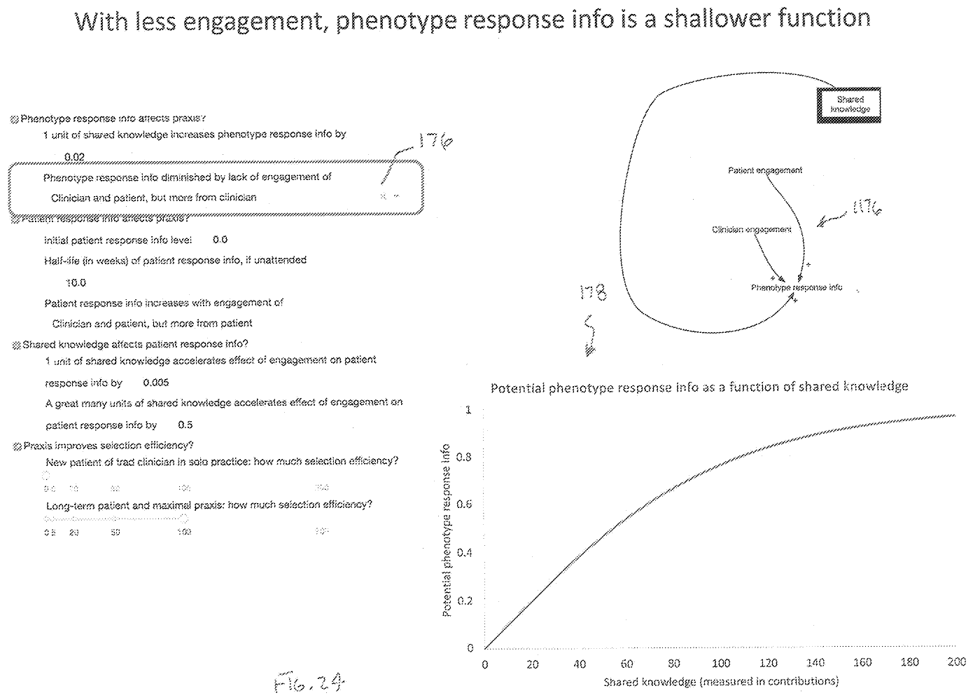

As shown in FIG. 24, menu 176 allows the user to indicate whether the phenotype response information may be diminished by lack of engagement, for example, between clinician and patient. In this example, if there is less than optimal engagement from a particular clinician and patient pair, there will be a smaller accretion of phenotype response information. The amount is a weighted mean of the clinician engagement and the patient engagement on a zero to 1 scale for each of them. In this example, it's weighted more toward the clinician. See 1176 in the diagram.

As an example, in graph 178, a participating patient and participating clinician are both at 50% toward fully engaged. That's going to reduce the initial slope by 50%. The notion is they are less engaged and so they're getting less from the shared knowledge and ultimately, if they're not engaged, they're not getting anything from the shared knowledge.

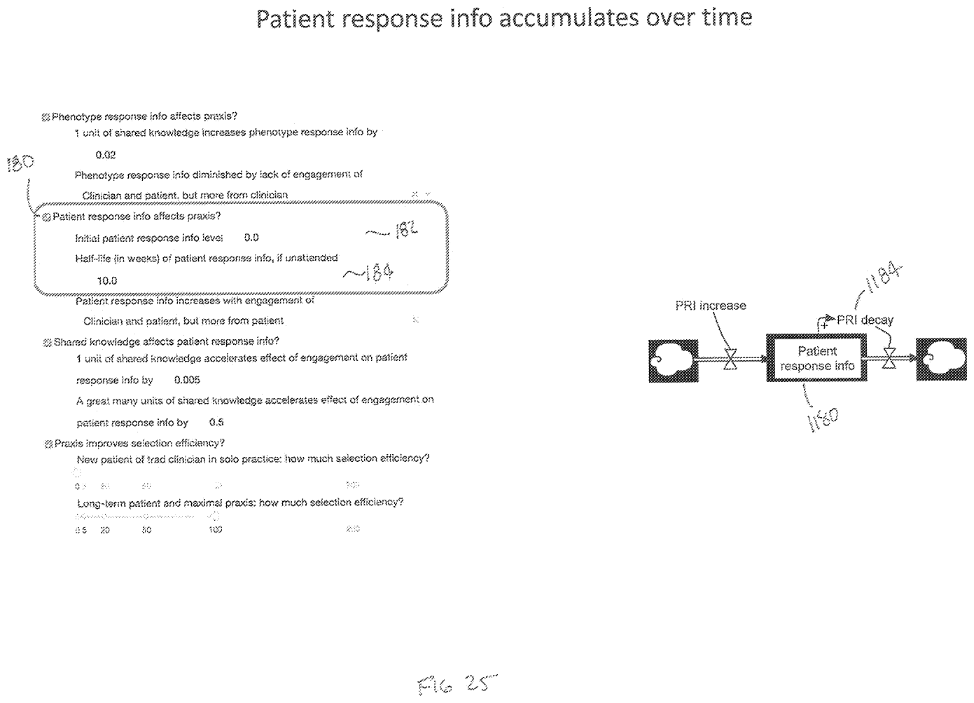

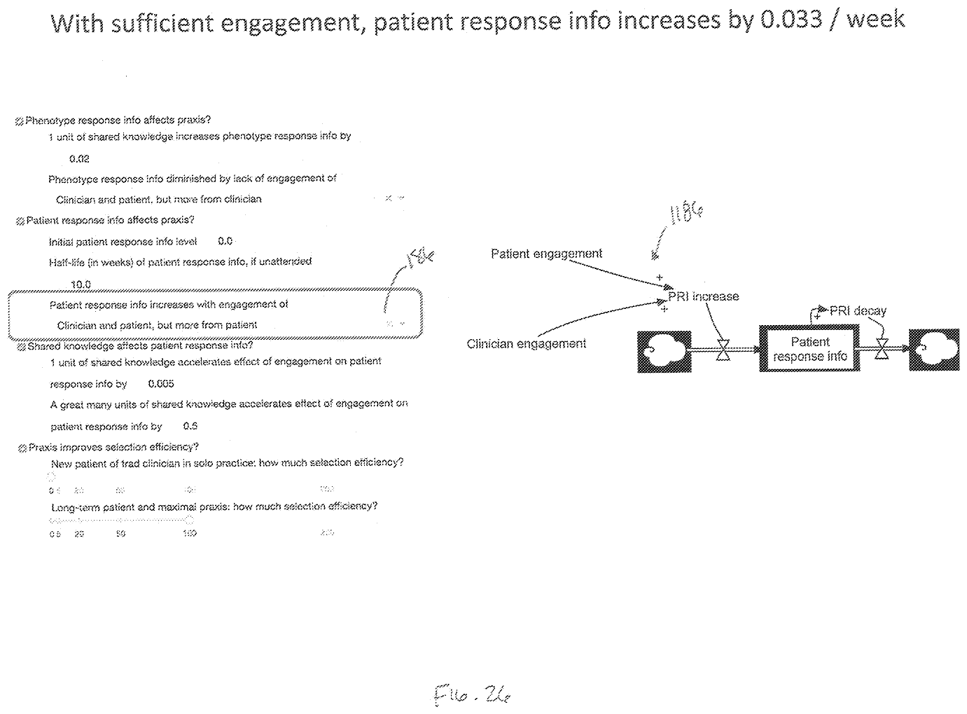

Referring to FIG. 25, check-box 180 is activated if patient response information affects praxis. Patient response information is a little bit different because it actually accumulates over time as it's collected. Thus, there is an initial level set in field 182 and then a half-life decay if patient response information is unattended set in field 184. If the patient stops paying attention, then that's going to decay over time because information from a couple of months ago is less relevant. As shown in FIG. 26, menu item 186 defines how the patient response information increases, for example, as a function of patient engagement and clinician engagement. Again, may be weighted in some way by default more from the patient. For example, if they're fully engaged right now, it's going to increase the patient response information by 0.033 a week. With a 10-week decay and 0.033 a week and fully engaged patient information, they'd ultimately get to about one. If they're less engaged, it increases slower and given the half-life, it probably won't ultimately get to patient response information of one. Notice that this 0.033 a week is actually not exposed right now as a model parameter in this user interface example.

In one example, patient response information could be defined as a set of questions the clinician asks the patient. Then, exemplary models can be run on different sets of questions that may be asked of the patients. Then, based upon what set of questions modeled best, the actual clinicians can be advised.

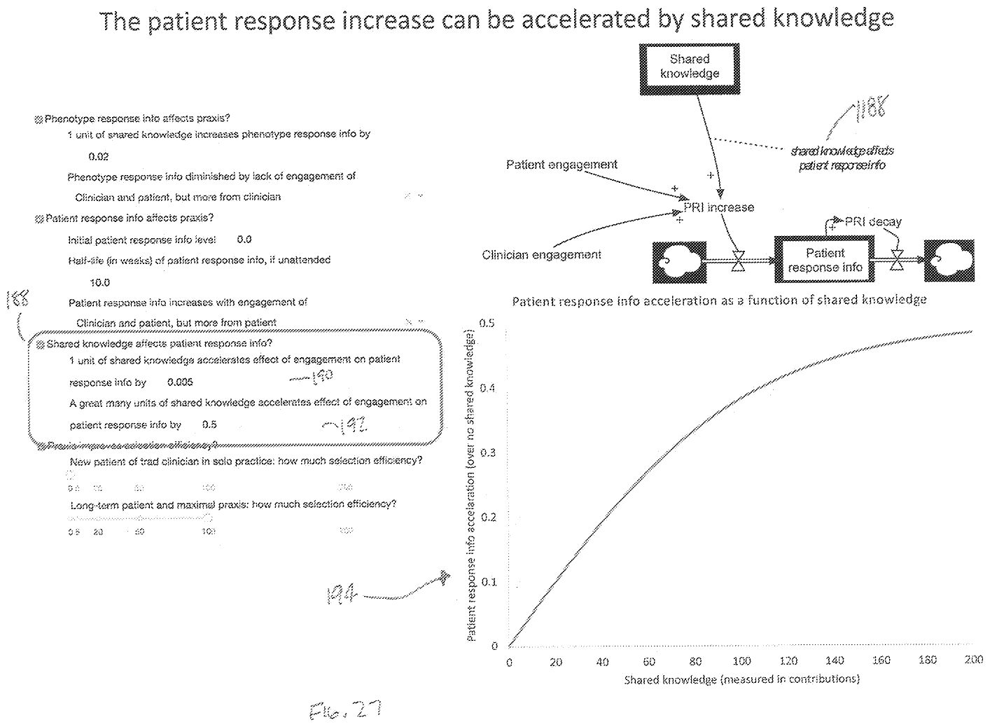

Referring to FIG. 27, a checkbox 188 is activated if shared knowledge (knowledge from the network) affects patient response information. Shared knowledge could include tools for helping patients collect patient response information, for example. It operates by accelerating the increase of patient response information. In field 190, the user can set a value of how much 1 unit of shared knowledge accelerates effect of engagement on patient response information, and field 192 indicates if there is a large number of shared units what is the total acceleration effect of such engagement on patient response information. In the example shown in FIG. 27 one unit of shared knowledge will accelerate it by half of 1% (field 190) and even if there is a lot of shared knowledge, it's only going to accelerate it by 50% (field 192). This example is reflected by graph 194.

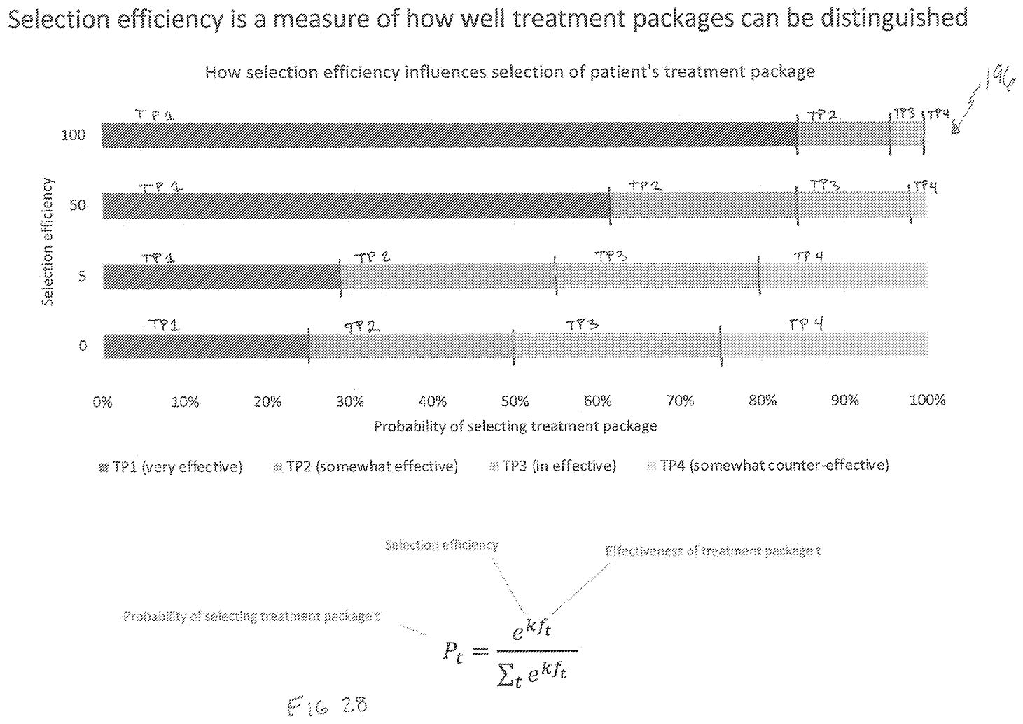

FIG. 28 illustrates Selection Efficiency. Selection efficiency is a measure of how well treatment packages can be distinguished. Graph 196 illustrates how selection efficiency allows a clinician to distinguish between different treatment packages of different effectiveness for particular phenotype. In this example, treatment package (TP) 1 through 4 are, respectively, very effective, somewhat effective, ineffective, and somewhat counter-effective. With zero selection efficiency, the chances of selecting each of these is about equal. As selection efficiency increases (in the example, to 5, 50, or 100), the chances of selecting a more effective treatment package increases and the chances of selecting an ineffective or counter-effective treatment package is reduced.

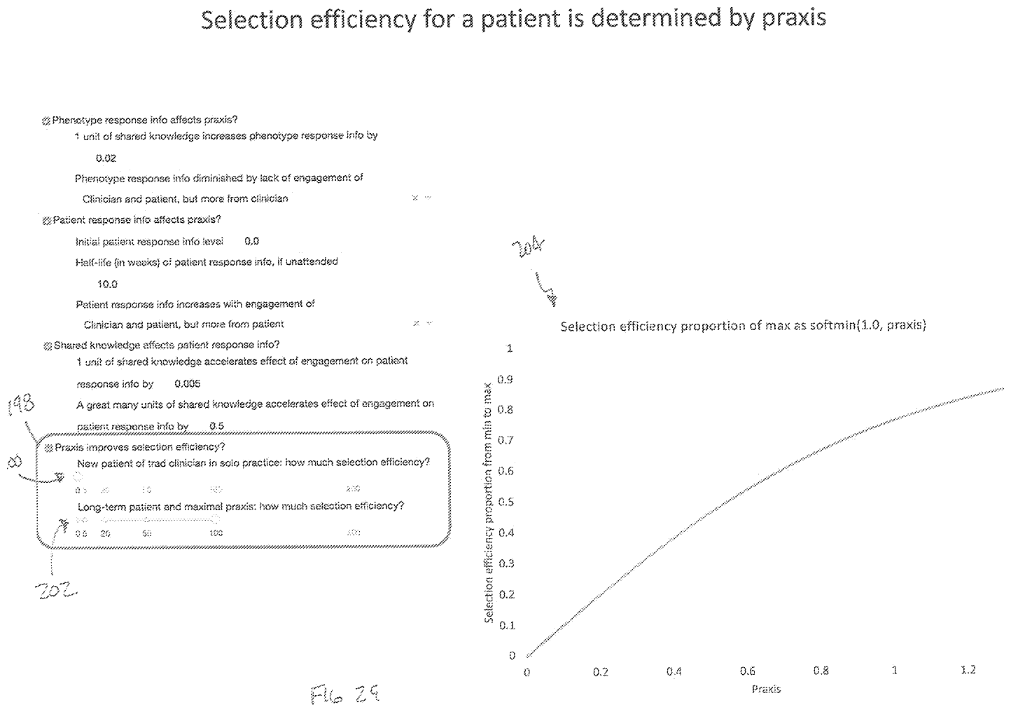

As shown in FIG. 29, a checkbox 198 is provided to indicate whether praxis improves selection efficiency. Sliders 200 and 2002 set the upper and lower limit of the selection efficiency. Slider 200 sets the amount of selection efficiency for a new patient of a traditional clinician in solo practice (in this case, zero, but it doesn't have to be zero). Slider 202 sets how much selection efficiency for a long-term patient and maximal praxis. Referring to the graph 204, the zero is the minimum, whatever the minimum happens to be, the one is this maximum, whatever the maximum happens to be, and then the praxis will determine how far it can get to the maximum.

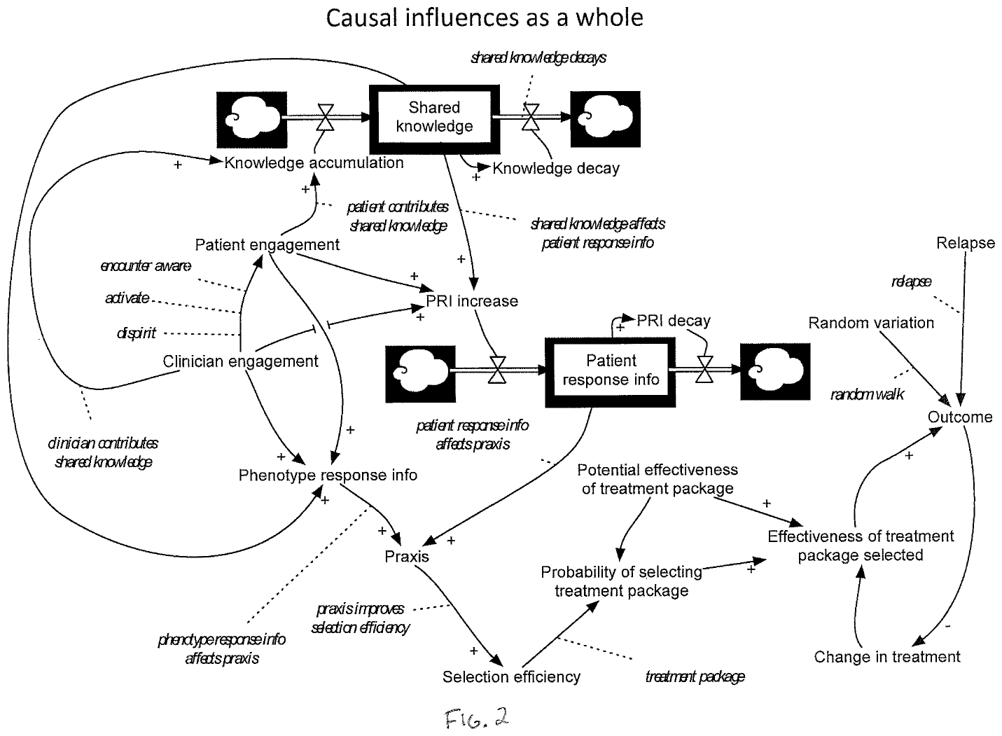

FIG. 2 illustrates the entire model. Items (x-y), are the parameters that can be shut off and on, and adjusted.

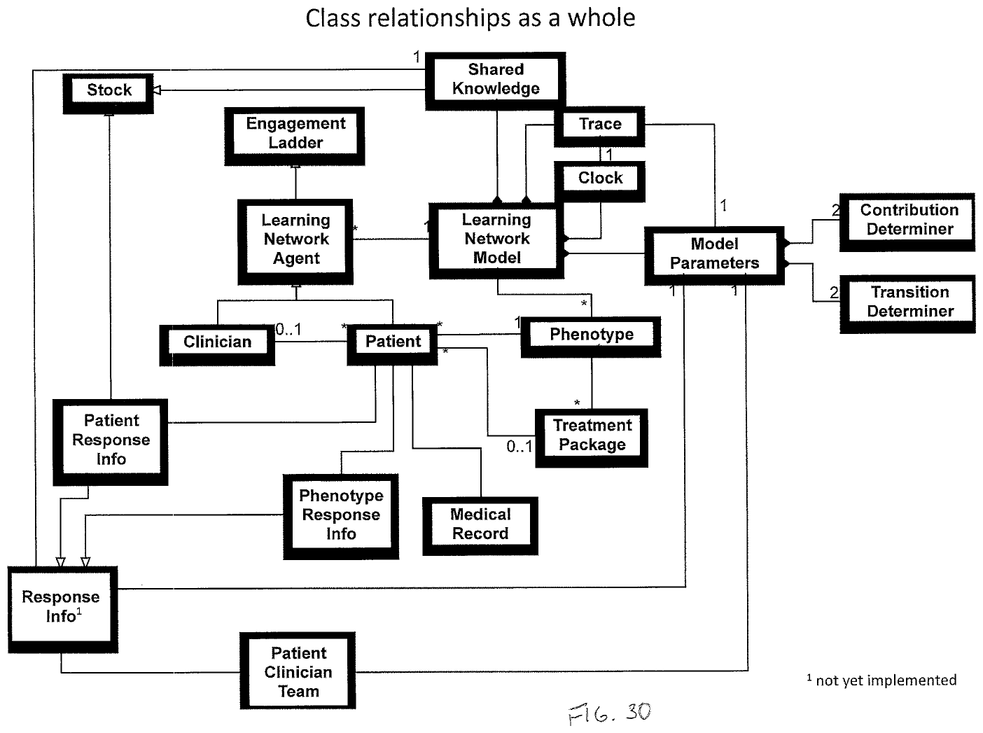

FIG. 30 illustrates the relationships among class entities within the model, including agents (patients, clinicians) and states, the effect of multiple interactions on shared knowledge, the various types of information and sources, and the parameters for these.

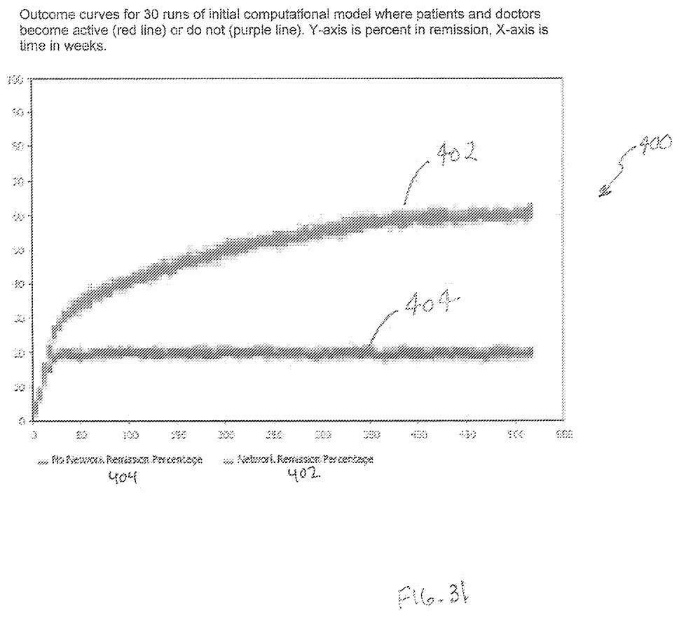

FIG. 31 is an example outcome curve 400 that may be displayed upon adjusting various parameters and ran. The example outcome curve in FIG. 31 is for 30 runs of an initial computational model where patients and doctors become active (line 402) and where patients deo not become active (line 404). The Y-axis is percent in remission, and the X-axis is time in weeks.

While various aspects and embodiments have been disclosed herein, other aspects and embodiments will be apparent to those skilled in the art. The various aspects and embodiments disclosed herein are for purposes of illustration and are not intended to be limiting.

What is claimed is:

1. A system for transforming characteristics of a patient agent and characteristics of a clinician agent into a visual representation for improved understanding of how interactions between the patient agent and the clinician agent change patient agent outcomes comprising instructions stored on a non-transitory memory and executable by a processor, the instructions comprising:
accepting inputs corresponding to the characteristics of the patient agent and the characteristics of the clinician agent, the inputs including a patient agent state of engagement for the patient agent and a clinician agent state of engagement for the clinician agent;
firstly simulating how the patient agent and the clinician agent interact with respect to information concerning a first treatment selection and a first treatment efficacy, the patient agent state of engagement, and the clinician agent state of engagement;
recording how the patient agent and the clinician agent interact in the firstly simulating step; and
secondly simulating additional, iterative interactions between the patient agent and the clinician agent with respect to an outcome of the first treatment selection, the patient agent state of engagement, and the clinician agent state of engagement;
recording how the interactions between the patient agent and the clinician agent change outcomes over time;
providing a visual representation of the changing outcomes;
comparing the changing outcomes to an actual learning network; and
manipulating the actual learning network based on the comparison.

2. The system of claim 1, the instructions further comprising:
thirdly simulating iterative interactions between the patient agent and the clinician agent with respect to information concerning a second treatment selection and a second treatment efficacy; and
recording how the interactions between the patient agent and the clinician agent from the thirdly simulating step change outcomes over time.

3. The system of claim 1, the instructions further comprising:
providing a user interface allowing a user to modify one or more key drivers and to view how such modifications change outcomes over time.

4. The system of claim 3, wherein key drivers include (1) access to information and (2) sharing information.

5. The system of claim 1, the instructions further defining two modules comprising:
a generic core module representing one or more adjustable factors determining patient-treatment matching; and;
a condition-specific module representing the impact of patient-treatment matching on patient-level outcomes.

6. The system of claim 5, wherein the output from the generic core module is represented as knowledge for matching patients to treatments and serves as an input into the condition-specific module.

7. The system of claim 6, wherein the generic core module represents both an initial patient-treatment matching and one or more iterative improvements of the patient-treatment matching.

8. The system of claim 6, wherein the model is built up from iterative interactions between patient agents and clinician agents.

9. The system of claim 8, further comprising instructions for accepting parameters, the parameters comprising:
the numbers of patient agents and clinician agents;
one or more patient agent characteristics including a patient phenotype, a degree to which a patient agent is informed, or a degree to which a patient agent is activated;
one or more clinician agent characteristics including a degree to which a clinician agent is prepared or a degree to which a clinician agent is proactive;
one or more rules under which one or both of the patient agent characteristics and the clinician agent characteristics change; and
an initial network structure among the clinician agents and the patient agents.

10. The system of claim 9, wherein the one or more rules comprises one or more of patient agents becoming more activated when exposed to a peer network or patient agents becoming more activated when interacting with a prepared and proactive clinician agent.

11. The system of claim 9, wherein the initial network structure includes multiple patient agents linked many-to-one to a first clinician agent to simulate a patient panel.

12. The system of claim 9, wherein the parameters further include one or more inputs defining a commons, wherein the inputs defining the commons include how much information is available, the rate at which information generated at the point of care is captured, or the rate at which captured information is sharable.

13. The system of claim 12, wherein the parameters further include one or more inputs defining collaboration between the patient agents and the clinician agents including governing how often patients and clinicians interact, one or more rules for determining how and how much information is produced at each clinical interaction, a rate at which information is spread across patient-patient networks and clinician-clinician networks, or the rate at which information is reliably implemented into the chosen patient-treatment match.

14. The system of claim 13, wherein the patient agents vary along multiple characteristics including the phenotype of the condition, the severity of the condition, a level of engagement, a level of adherence to treatment, a response to treatment, a degree of learning from other patients, and the arrival to and departure from the learning network; and
wherein the clinician agents vary along multiple characteristics including a level of engagement, an ability to correctly diagnose a patient agent condition, a degree of learning from patient agents, a degree of learning from other clinician agents, and a level of employment turnover.

15. The system of claim 14, wherein the inputs governing how often the patient agents and the clinician agents interact include one or more selectable events including an initial diagnosis, a treatment prescription, a monitoring stage, a subsequent diagnosis, and an adjustment in treatment.

16. The system of claim 15, wherein the instructions are executable on a processor of a local machine or a cloud-based architecture employing one or more multi-thread processors enabling different users to utilize the tool to enter data and visualize results simultaneously from different locations.

17. The system of claim 15, the instructions further comprising:
computing scenario-specific learning collaborative outcome metrics; and
presenting the results of the computing step to users visually and as a data file.

18. The system of claim 12, the instructions further comprising:
accepting one or more inputs corresponding to critical collaborative parameters including:
a number of patient agents;
a number of clinician agents;
one or more rules by which:
patient agents and clinician agents interact and the effect of the interactions on patient agent states and clinician agent states;
how patient agents and clinician agents produce knowledge for making decisions and the effect of those decisions on treatments and outcomes; and
how and under what circumstances knowledge is shared; and
providing one or more outcome parameters including individual and median patient agent outcomes over time, proportion of patient agents above a certain threshold, time between patient agent presentation and relief of symptoms, and time between periods of disease exacerbation.

19. The system of claim 15, the instructions further comprising presenting one or more visualizations of model variables, the visualizations including one or more of graphs of variables as a function of time, or a phase diagram of outcome end-points as a function of parameter settings.

20. The system of claim 19, further comprising instructions accounting for variation and uncertainty in input parameters and illustrating uncertainty bounds in output visualizations.

21. The system of claim 1, wherein the manipulating includes promoting one or more of a patient and a clinician to contribute to the actual learning network.

22. The system of claim 1, wherein recording how the interactions between the patient agent and the clinician agent change outcomes over time comprises determining phenotype response information and the phenotype response information is based on a response of one or more phenotypes to the first treatment.

23. The system of claim 1, wherein recording how the interactions between the patient agent and the clinician agent change outcomes over time comprises determining patient response information and the patient response information is based on the outcome of the first treatment selection.

24. The system of claim 23, wherein the patient response information increases as a function of the patient state of engagement.

25. The system of claim 23, wherein the patient response information increases as a function of the clinician state of engagement.

26. The system of claim 23, wherein the patient response information decreases as a decaying function over time.
